(19) United States
(12) Patent Application Publication
(10) Pub. No.: US 2012/0244234 A1
(43) Pub. Date: Sep. 27, 2012

(54) NIGELLA EXTRACTS FOR THE TREATMENT OF SYMPTOMS CONNECTED TO IMPAIRED OR IMBALANCED NEUROTRANSMISSION

(76) Inventors: Stephane Etheve, Village-Neuf (FR); Ann Fowler, Rheinfelden (CH); Regina Goralczyk, Grenzach-Wyhlen (DE); Claus Kilpert, Mannheim (DE); Hasan Mohajeri, Egg b. Zuerich (CH); Loni Schweikert, Wallbach (CH)

(21) Appl. No.: 13/386,471

(22) PCT Filed: Jul. 20, 2010
(86) PCT No.: PCT/EP10/60485

§ 371 Date: Jun. 7, 2012

(30) Foreign Application Priority Data
Jul. 21, 2009 (EP) 09166018.3

(51) Int. Cl. A61K 36/71; A61P 25/20; A61P 25/22; A61P 25/24; A61P 25/00; A61P 25/18

(52) U.S. Cl. 424/725

(57) ABSTRACT

The present invention relates to the use of Nigella sp. extracts and their volatile components for the treatment of symptoms connected to impaired neurotransmission in animals including humans as well as to dietary supplements, food and feed or nutraceutical compositions containing such extracts or their volatile components.

### FIELD OF THE INVENTION

The present invention relates to the use of lipophilic Nigella sp. seed extracts as agents for the treatment of symptoms connected to impaired, i.e. reduced neurotransmission. In addition, it also relates to dietary compositions containing such Nigella sp. seed extracts.

### BACKGROUND OF THE INVENTION

Nigella sativa is commonly called fennel flower, nutmeg flower, Roman coriander, blackseed, black carroway, black onion, or black cumin (although Bunium persicum is also referred to as black cumin).

Traditionally, Nigella sativa has been thought of as having anti-hypertensive properties, carminative properties (i.e. preventing/removing gas in the gastrointestinal tract) or anti-helminic properties. Total seed extracts have also been traditionally used to stimulate the body's energy and help recovery from fatigue and dispiritedness.

El-Hadiyah et al 2003 Natural Prod. Sci 9(1):22-27 describe the chemical makeup of Nigella sativa oil. Total oil contains two types of oils: fixed oil and volatile oil. Volatile oil contains thymoquinone and other monoterpenes. Total oil was reported to have analgesic, antidepressant, and central nervous system sedative activity.

Perveen et al 2009 Pak. J. Pharm. Sci. 22(2):139-144 describe anti-anxiety effects and an increase in 5-HT (serotonin) levels in the brain upon oral administration of Nigella sativa oil. The type of oil was not specified.

Raza et al 2006 J. Herbs, Spices & Med. Plants 12(1/2):153-164 tested anti-anxiety effects of i.p. injections of Nigella sativa volatile oil, fixed oil, aqueous extracts and single constituents. In the Y-maze test all were positive; and for hole-board test, only the aqueous extract gave negative results. In the water-lick test, only volatile oil was positive.

Kandill US Patent Publication 2005/0214393 discloses Nigella sativa lipid fractions containing polyunsaturated fatty acids, saturated fatty acids, glyceryl esters, volatile oils and sterols. This is useful for a variety of topical applications.

### DETAILED DESCRIPTION OF THE INVENTION

It has been surprisingly found that lipophilic extracts of Nigella sp., especially Nigella sativa seeds, administered orally are serotonin re-uptake inhibitors, thus prolonging the time that serotonin is available for neurotransmission. Thus, food, feed, and/or nutraceutical compositions containing these extracts are particularly useful for treating mood related symptoms which are mediated by misbalanced serotonin neurotransmission or a misbalanced serotonin neurotransmission in combination with noradrenalin and dopamine neurotransmission in individuals who are otherwise mentally healthy.

Such misbalanced serotonin neurotransmission may occur under certain circumstances in normal healthy people eg, under acute or chronic stress, after nights of bad sleep or sleeplessness, shift work or jet lag. It may also occur in people who are not clinically diagnosed for depression, but susceptible for mood or emotional imbalances/instabilities, winter blues, extensive worrying, pessimism, psychic pressure (eg under divorce, unemployment), or a general melancholic temper.

This invention also relates to the use of lipophilic Nigella sp., especially Nigella sativa seed extracts which do not contain sterols, for the manufacture of compositions for the treatment and prevention of symptoms in healthy individuals connected to impaired or reduced neurotransmission. Thus, in one aspect the present invention relates to dietary compositions comprising at least one lipophilic Nigella sp., especially Nigella sativa seed extract for the treatment of symptoms connected to impaired or reduced neurotransmission in otherwise healthy individuals.

Another aspect of this invention is a method of treating or preventing the occurrence or severity of a mood condition connected to impaired or reduced neurotransmission in an individual who has otherwise normal mental health, comprising orally administering an effective amount of a lipophilic Nigella sp. extract, and observing the prevention or reduction in severity of the condition.

### BRIEF DESCRIPTION OF THE FIGURES

FIG. 1 shows the measured IC₅₀ values for inhibition of serotonin uptake by Nigella sativa lipophilic extract is 14.14±4.5 μg/mL.

FIG. 2 shows the IC₅₀ values for inhibition of serotonin uptake by a cold-pressed Nigella sativa extract. As can be seen, cold-pressed Nigella sativa extract does not inhibit serotonin uptake.

FIG. 3 shows a chromatogram of a Nigella sativa extract containing fatty acids and 0.01% (w/w) thymoquinone.

The main neurotransmitters are serotonin, dopamine, noradrenaline, acetylcholine, glutamate, gamma-amino-butyric acid. Those neurotransmitters of particular relevance to mood-related symptoms are serotonin, noradrenaline, and dopamine. Increase in neurotransmission is achieved by increasing the concentration of the neurotransmitter in the synaptic cleft thus making it available for increased or prolonged neurotransmission through inhibition of re-uptake into the pre-synaptic nerve end, or by preventing neurotransmitter catabolism by inhibition of degrading enzymes such as monoamine oxidase A and B.

### DEFINITIONS

The terms “impaired neurotransmission” and “reduced neurotransmission” are used interchangeably throughout the present application. They are used in the present application in accordance with their meaning well-known to the person skilled in the art, and relate to a state of deregulation of neurotransmission, which may occur at the level of neurotransmitter biosynthesis, processing, storage, release, re-uptake and receptor binding. Impaired neurotransmission, in particular a reduction of neurotransmission, may manifest itself in animals including humans which are otherwise mentally healthy as a disturbance of emotions, or mood. Such disturbancenes may occur under certain life conditions in normal healthy people eg, under acute or chronic stress, after nights of bad sleep or sleeplessness, shift work or jet lag. It may also occur in people not clinically diagnosed for depression, but susceptible for mood or emotional imbalances/instabilities, winter blues, extensive worrying, pessimism, psychic pressure (eg under divorce, unemployment), or a general melancholic temper.

The term “lipophilic seed extract” means that the seeds have been extracted using a lipophilic organic solvent which is suitable (i.e. approved by regulatory agencies) for food use, or alternatively, a water-based distillation which yields essential oils. Examples of suitable organic solvents include: liquid carbon dioxide under supercritical conditions “SFCO2”, ethanol, ethyl acetate, methyl-tert.-butylether/methanol, propane, butane, acetone and nitrous oxide. These extracts can be made using known techniques, and typically contain a variety of potentially active ingredients, such as: nonterpenoid hydrocarbons, monoterpenoid hydrocarbons, monoterpenoid ketones, monoterpenoid alcohols, sesquiterpenoid hydrocarbons and phenyl propanoid compounds. “Lipophilic seed extract” as used herein, specifically excludes extracts which contain only the so-called fixed oil fraction, which are obtained by cold-pressed methods and which contain only fatty acid components. It also excludes extracts containing sterols.

“Nigella sp.” may include any member of the Nigella species. A preferred plant is Nigella sativa, but other species of Nigella may also be used, such as N. damascena L.

“Animals” includes humans, and encompasses mammals, fish and birds. Preferred are: humans, pets or companion animals, farm animals, and animals used in the fur industry.

“Farm animals” includes: fish, such as salmon and trout, aquaculture animals such as shrimp, pigs, horses, ruminants (cattle, sheep, goats) and poultry (such as geese, chickens, broilers, laying hens, quails, ducks, and turkeys). Preferred are poultry, cattle, sheep, goats and pigs.

“Pets” or “companion animals” include dogs, cats, birds, aquarium fish, guinea pigs, (jack) rabbits, hares and ferrets. Dogs and cats are preferred.

“Animals used in the fur industry” include minks, foxes, and hares.

“Dietary compositions” includes any type of nutritional product, such as (fortified) food/feed and beverages, and also includes clinical nutrition products, and dietary supplements.

“Fortification” means that a Nigella sp., preferably a Nigella sativa extract was added during manufacture of the food/feed or beverage.

“Prevent” does not mean that a symptom will never appear. Rather, it refers to: prophylactic treatments which delay the onset of symptoms, and/or reduce the severity or frequency of symptoms if they appear, early intervention, and generally lessening the risk of symptoms or conditions.

“Observing” can be done by either the individual who was administered the composition, or a third party. It may include an objective measurement, or may be a subjective comparison.

The target population for food/feed/neutraceuticals/food supplements of this invention are individuals who are mentally healthy, i.e. have not had any psychiatric diagnosis or tendencies towards clinical depression, anxiety or the like. They are generally of sound mind, although may be subjected to everyday stress. As for animals, the population is not known to be particularly aggressive or otherwise exhibit noteworthy negative behaviors.

The compositions of this invention can thus be characterized as mood balancing agents, mood/vitality improvers, stress relievers, tension reducers, relaxants, sleep improvers, and normalizers of biorhythms.

Numerous pharmaceutical compositions which are neurotransmitter regulators have proven helpful in various mood-related symptoms. As the extracts of this invention have been found to work using the same or similar biochemical pathways, then it can be concluded that they are useful for similar conditions.

Attention Deficit Hyperactivity Disorder (ADHD)

A number of antidepressants which affect reuptake of one or more of the monoamines are also effective in the treatment of ADHD. However, many of the children or adults with ADHD are not clinically and mentally ill, but need sustained, chronic support for their brain neurotransmitter function over their life-time. They would benefit from a mild, chronic intake of a moderately acting nutraceutical instead of chronic intake of drugs with side effects.

Circadian Rhythm Disturbances

Mood related symptoms of impaired neurotransmission and occupational stress can lead to disturbances in circadian rhythms (so-called biorhythms). These conditions are often chronic and persistent. Also, deregulation of circadian rhythms induced by long-distance flights (jet-lag), as well as by shift-work, can cause similar symptoms and distress. Therefore, treatment with dietary supplementation to maintain the normal circadian rhythm (that an animal or human is used to), and/or to alleviate and prevent symptoms associated with a disturbed circadian rhythm, such as impairment of cognitive function and memory, and mental and physical fatigue, is warranted to improve the overall quality of life and to benefit the vital energy of a person in need thereof

Sleep Disturbances, Insomnia, and Chronic Fatigue Syndrome

Low mood, mood shifts and sleep disturbances are closely linked. This has been observed in clinically depressed patients. It can be assumed that similar mechanism occur in the non-clinically depressed person at conditions of low mood and stress. Thus, the compositions of this invention may help to alleviate mood associated sleep disturbances via the neurotransmitter balancing action.

Similary, as TCAs are commonly used for treatment of chronic fatigue syndrome, the composition of this invention due to a similar mode of action, but less side effects, may be favourable over drugs for the sustained and long-erm alleviation of symptoms as it is required in these persons.

Pain

Antidepressants with differing mechanisms of action can also be effective in the treatment of pain. For example, amitriptyline and mianserin, which are potent 5-HT₂ antagonists, are used for the control of chronic pain [Blier, et al 2001. J. Psych & Neuro, 26(1): 37-43]. The noradrenaline/serotonin/dopamine reuptake inhibitor, duloxetine, is efficacious in the treatment of neuropathic pain associated with diabetic peripheral neuropathy [Chouinard, 2005. J. Psych & Neuro. 31(3): 168-176]. This type of pain is different from that associated with inflammation. According to this invention, pain is reduced using a different mechanism than decreasing inflammation.

Thus, the invention relates to a method for the treatment or prevention of a symptom connected to impaired neurotransmission, said method comprising orally administering an effective amount of a lipophilic Nigella sp, or preferably a Nigella sativa seed extract to an animal (including humans) which is in need thereof, and observing an improvement in the symptom connected to impaired neurotransmission.

The lipophilic Nigella sp. and preferably Nigella sativa seed extracts can be used for the manufacture of nutraceuticals, nutritional supplements or dietary supplements for the treatment of a condition connected to impaired or imbalanced neurotransmission. They can additionally be used for the manufacture of compositions for use as mood balancing agents, mood/vitality improvers, stress relievers, condition improvers, reducers of tension, reducers of sadness, reducers of unhappiness/discontent, reducers of irritability, reducers of dysphoria, reducers of obsessive-compulsive behaviour, relaxants, sleep improvers and/or insomnia alleviators.

“Mood improver”, “vitality improver” or “emotional wellness booster” means that the mood or vitality of a healthy person treated with it is enhanced, that his/her self esteem is increased and/or that negative thoughts and/or negative tension, sadness, unhappiness/discontent and irritability, and dysphoria are/is reduced. It also means that emotions are balanced and/or that the general, especially the mental, well-being and vitality is improved or maintained, as well as that the risk of mood swings is lessened, that the positive mood is retained, and that one feels energetic and motivated.

“Tension reducer, sadness reducer, unhappiness/discontent reducer, irritability reducer, dysphoria reducer” means that (chronic) tension and worrying are reduced or alleviated. Hypervigilance syndrome, including restlessness and muscle tension, are also reduced or relieved. Social and other phobias are also at least partially resolved. In general, the social environment is experienced as less threatening. The person is emotionally relaxed, experiences comfort and enjoys company and contact with other people. In general, the person feels energetic and motivated to conduct daily tasks.

A “relaxant” works by completely or partially correcting a person's circadian rhythm (biorhythm) which has been disturbed due to jet-lag or shift work. A relaxant will at least partially prevent or abolish the symptoms associated with such disturbances, i.e. impairment of cognitive function and memory, mental and physical fatigue, and improve overall quality of life and vital energy. Thus, the lipophilic Nigella sp. preferably Nigella sativa seed extracts may also be used to prevent and/or abolish impairment of cognitive function and memory, to prevent and/or abolish mental and physical fatigue, and to improve overall quality of life and vital energy.

A further embodiment of the present invention relates to the use of lipophilic Nigella sp., preferably Nigella sativa seed extracts and to the use of compositions containing them as “condition improvers”, i.e. as means to reduce irritability and tiredness, to reduce, prevent or alleviate physical and mental fatigue, to favour undisturbed sleep, that is to act against insomnia and sleep disturbances and to improve sleep, and to increase energy in more general terms, especially to increase brain energy production, in normal healthy individuals. Moreover, such “condition improvers” are to be used for cognition improvement in general, and especially for maintenance or improvement of attention and concentration, of memory and of the capacity for remembering, of learning ability, of language processing, of problem solving and of intellectual functioning; for improvement of short-term memory; for increasing mental alertness; for increasing the ability to focus and mental sharpness, for enhancing mental vigilance; for reducing mental fatigue; for supporting cognitive wellness, for maintaining balanced cognitive function, for the regulation of hunger and satiety as well as for the regulation of motor activity in normal healthy individuals.

Thus, a preferred aspect the invention relates to the use of lipophilic Nigella sativa seed extracts as mood balancing agents and/or stress relievers in mentally healthy individuals.

In a further preferred embodiment of the present invention the lipophilic Nigella sativa seed extracts are used for maintaining circadian rhythms in humans, for alleviating and/or for preventing the symptoms associated with a disturbed circadian rhythm in humans. Thus, mood is stabilized and an emotional balance is achieved to cope with daily life stress and to maintain physical and psychological performance. Furthermore, the symptoms associated with a disturbed circadian rhythm, such as impairment of cognitive function and memory, and mental and physical fatigue, are alleviated and/or prevented so that the overall quality of life is improved. These persons also benefit from maintaining vital energy. Also, deregulation of circadian rhythms induced by long-distance flights (jet-lag) as well as by shift-work and the symptoms associated with it are alleviated and/or prevented.

Another preferred aspect of the invention relates to the use of the lipophilic Nigella sativa seed extracts for the manufacture of a composition, to maintain or enhance mood status and/or maintain a healthy mood in individuals which are mentally healthy.

In another embodiment, an effective dose of lipophilic Nigella sp., preferably Nigella sativa seed extracts may especially be used by healthy individuals to help maintain mental well-being, for maintaining a balanced cognitive function, for helping to retain a positive mood, relaxation and for supporting cognitive wellness.

Veterinary Uses

Another aspect of this invention are veterinary uses of the lipophilic Nigella sp., preferably Nigella sativa seed extract. Animals may exhibit adverse behavioral and or physiological reactions to stressful situations. For example, animals raised in mass production environments, or being transported, can have a decline in meat or milk quantity or quality. Stressed poultry can resort to feather picking, reduced egg laying and cannibalism. Many animals can become aggressive or display obsessive-compulsive behaviors. The extracts of this invention, by acting on neurotransmitters, can relieve these unwanted behaviours and physiologies in animals.

In a preferred embodiment of the present invention the lipophilic Nigella sativa seed extracts are administered for preventing stress in farm animals, in mass production livestock husbandry, during transport to slaughter and/or for preventing quality loss of meat of said farm animals during transport to slaughter.

In another preferred embodiment of the present invention the lipophilic Nigella sativa seed extracts are administered to pets or companion animals for reduction of stress, tension and aggressiveness and compulsive behavior exhibited under stressful conditions, such as separation, change or loss of the owner, during holiday separation and husbandry in so called “animal hotels”, husbandry in animal shelters or refuges.

Pet animals and farm animals can be in conditions which particularly benefit from enhanced or improved neurotransmission. Such conditions e.g. occur after capture or transport or with housing.

Another embodiment of this invention is method for preventing stress in farm animals in mass production livestock husbandry, during transport to slaughter and/or for preventing quality loss of meat of said farm animals during transport to slaughter, comprising administering an effective dose of a lipophilic Nigella sativa seed extract to farm animals which are in need thereof, and observing a decrease in stress or an improvement in meat quality. The farm animals are preferably poultry, cattle, sheep, goats and swine.

Another aspect of this invention is a method for preventing and/or alleviating stress in aquaculture comprising the step of administering an effective dose of a lipophilic Nigella sativa seed extract to animals which are in need thereof, wherein the animals are fish or shrimp, and observing the effects of stress alleviation.

Yet another aspect of this invention is a method for reducing stress, tension, aggressiveness and/or compulsive behavior in pet animals under stressful conditions, such as separation, change or loss of the owner, during holiday separation and husbandry in so-called “animal hotels”, husbandry in animal shelter stations and other conditions of dense in husbandry and breeding, comprising the step of administering an effective dose of a lipophilic Nigella sativa seed extract to pet animals which are in need thereof, especially to cats and dogs which are in need thereof, and observing the reduction in stress, tension or agressiveness.

Still another aspect of this invention is a method for preventing and/or reducing symptoms associated with stressful conditions in animals used for the fur industry, preferably for minks, foxes and/or hares by administering a lipophilic Nigella sativa seed extract, and ovserving the reduction of stress.

For animals, the lipophilic Nigella sativa seed extracts are in preferably administered as fortified feed or fortified beverages (e.g. as addition to the drinking water).

Nigella sativa Extracts

Lipophilic Nigella sativa seed extracts may be obtained in accordance with methods well-known in the art, e.g., by (an) extraction with solvents like methanol, ethanol, ethyl acetate, diethylether, n-hexane, methylenechloride, or with supercritical fluids like carbon dioxide (pure or in mixture with other solvents such as alcohols) or dinitrogen oxide, (b) hydrodistillation for obtaining essential oils or (c) extraction/distillation with hot gases like nitrogen. They do not contain sterols.

The lipophilic Nigella sativa seed extracts can be of natural or synthetic or mixed (viz. partly natural, partly synthetic) origin, i.e., they can, apart from being obtained by extraction of plants and fractionation, be chemically synthesized and, if desired, mixed together in any desired quantities. They can be prepared and used in any desired purities and concentrations, e.g. as solutions containing them in concentrations as low as, e.g., 10% (w/w) or less, or up to nearly 100% (w/w).

Preferred are Nigella sativa extracts containing a high proportion of at least one of their volatile components. More preferred are Nigella sativa extracts containing at least a total of 70 weight-% of volatile components based on the total weight of the extract.

The composition of the present invention is preferably in the form of nutritional composition, such as fortified food, fortified feed, or fortified beverages, or in form of fortified liquid food/feed for animals including humans.

The dietary and nutraceutical compositions according to the present invention may be in any solid, semisolid or liquid galenic form that is suitable for administering to the animal body including the human body, especially in any form that is conventional for oral administration, e.g. in solid form, such as (additives/supplements for) food or feed, food or feed premix, fortified food or feed, tablets, pills, granules, dragées, capsules, and effervescent formulations such as powders and tablets, or in liquid form such as solutions, emulsions or suspensions as e.g. beverages, pastes and oily suspensions. The pastes may be encapsulated in hard or soft shell capsules, whereby the capsules feature e.g. a matrix of (fish, swine, poultry, cow) gelatin, polysaccharides like starches or pullulan, or cellulose derivatives like hydroxypropyl-methyl-cellulose. Examples for other application forms are forms for transdermal, parenteral or injectable administration. The dietary and nutraceutical compositions may be in the form of controlled (delayed) release formulations.

The dietary compositions according to the present invention may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents, gel forming agents, antioxidants and antimicrobials.

Examples of fortified foods are vegetable juices, spicy cereals, and bakery items, such as spicy cookies or breads. Examples of dietary supplements are tablets, pills, granules, dragées, effervescent formulations and in specific capsules, in the form of non-alcoholic drinks, such as vegetable juices or other drinks or teas, in the form of liquid food, such as soups.

Beverages encompass non-alcoholic and alcoholic drinks as well as liquid preparations to be added to drinking water and liquid food. Non-alcoholic drinks are e.g. soft drinks, sport drinks, vegetable juices (e.g. tomato juice), and teas. Liquid foods are e.g. soups.

In addition to Nigella sativa extract, the compositions according to the present invention may further contain conventional additives and adjuvants, excipients or diluents, including, but not limited to, water, gelatin of any origin, vegetable gums, ligninsulfonate, talc, sugars, starch, gum arabic, vegetable oils, polyalkylene glycols, flavoring agents, preservatives, stabilizers, emulsifying agents, buffers, lubricants, colorants, wetting agents, fillers, and the like. The carrier material can be organic or inorganic inert carrier material suitable for oral/parenteral/injectable administration.

For humans a suitable daily dosage of lipophilic Nigella sativa seed extracts or their volatile components for the purposes of the present invention may be within the range from 0.001 mg per kg body weight to about 100 mg per kg body weight per day. More preferred is a daily dosage of about 0.01 to about 50 mg per kg body weight, and especially preferred is a daily dosage of about 0.05 to 10.0 mg per kg body weight.

In solid dosage unit preparations for humans, the lipophilic Nigella sativa seed extract is suitably present in an amount from about 0.1 mg to about 6000 mg, preferably from about 1 mg to about 1000 mg, most preferably from about 25 mg to 500 mg per dosage unit. For relief of symptoms associated with conditions as mentioned herein, the lipophilic Nigella sativa seed extract is taken once or twice per day together with a meal for at least one week and up to 6-12 months. For prevention of occurrence of symptoms associated with conditions as mentioned herein and for the maintenance of a generally relaxed state, consumption on a regular basis is suitable.

In dietary compositions, especially in food and beverages for humans, the lipophilic Nigella sativa seed extract is suitably present in an amount of from about 0.0001 (1 mg/kg) to about 5 weight-% (50 g/kg), preferably from about 0.001% (10 mg/kg) to about 1 weight-%, (10 g/kg) more preferably from about 0.01 (100 mg/kg) to about 0.5 weight-% (5 g/kg), based upon the total weight of the food or beverage. For relief of symptoms associated with conditions as defined above, the food product is taken once or twice per day at least for one to three weeks or on a regular basis, i.e. at least once daily.

In food and drinks in a preferred embodiment of the invention the amount of the lipophilic Nigella sativa seed extracts is 10 to 30 mg per serving, i.e. 120 mg per kg food or drink. The food product is taken once or twice per day at least for one to three weeks or preferably on a regular basis of at least once daily.

For animals excluding humans, a suitable daily dosage of a lipophilic Nigella sativa seed extract may be within the range from 0.001 mg per kg body weight to about 1000 mg per kg body weight per day. More preferred is a daily dosage of about 0.1 mg to about 500 mg per kg body weight, and especially preferred is a daily dosage of about 1 mg to 100 mg per kg body weight. To prevent and reduce symptoms associated with stressful conditions, such as mass production livestock husbandry and fur industry husbandry or aquaculture, the product containing the lipophilic Nigella sativa seed extract is given over the animal's entire lifetime until slaughter. Especially in the case, where farm animals, such as poultry, cattle, sheep, goats and swine, especially cattle and swine, are transported to slaughter, they should be administered a daily dosage of about 3 to 800 mg/kg body weight of the extract, preferably during transportation, more preferably at least 3 days before transportation and during transportation.

For pets, under stressful conditions as in animal shelter farms or pet shops, the product should be given for at least 1-3 weeks, or over the whole husbandry period. Under conditions of short-term stress, such as holiday separation, husbandry in animal “holiday hotels”, visits to or stays in veterinarian clinics, the product may be given at least 3 days, and preferably 7 days, before the stressful event.

The invention is illustrated further by the following non-limiting examples.

### EXAMPLES

### Example 1

### Preparation of Nigella sativa Extracts

Lipophilic Nigella sativa seed extracts can be prepared from the seeds by hydro-distillation (steam distillation), extraction with organic solvents (such as but not limited to ethanol, ethyl acetate, or SF-CO₂) according to commonly available literature.

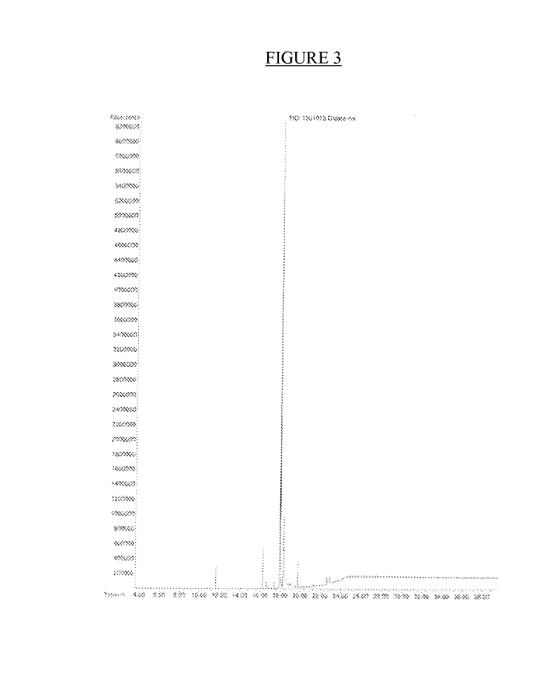

A GCMS chromatogram of a Nigella sativa extract from Analytikon and 0.1% (w/w) thymoquinone is shown in FIG. 3.

Extract I

10 g of Nigella sativa seeds were extracted with methyl-tert.-butylether/methanol (9/1) resulting in 2.2 g of a lipophilic extract.

### Example 2

### Serotonin Uptake Inhibition by Nigella sativa Extracts

HEK-293 cells stably expressing the human serotonin re-uptake transporter (hSERT) were obtained from R. Blakely, Vanderbilt University, USA. The cells were routinely grown in Dulbecco's Modified Eagle's Medium, purchased from Bioconcept, Allschwil, Switzerland containing 10% fetal calf serum, penicillin, streptomycin, L-glutamine and the antibiotic G418 and passaged by trypsinisation. 1 day prior to the assay cells were seeded in the above mentioned medium. Immediately prior to the assay the medium was replaced by Krebs-Ringer bicarbonate buffer, purchased from Sigma Chemicals Ltd., supplemented with 35 μM pargyline, 2.2 mM CaCl₂, 1 mM ascorbic acid and 5 mM N-2-hydroxyethyl-piperazine-N′-2-ethanesulfonic acid (“Hepes” buffer). Serotonin uptake into the cells was determined by addition of radio-labeled (³H) serotonin (Amersham Biosciences GE Healthcare, Slough, UK) to a concentration of 20 nM, and incubation for 30 minutes at room temperature. Following removal of unincorporated label by gentle washing three times with the above buffer, incorporated serotonin was quantified by liquid scintillation counting.

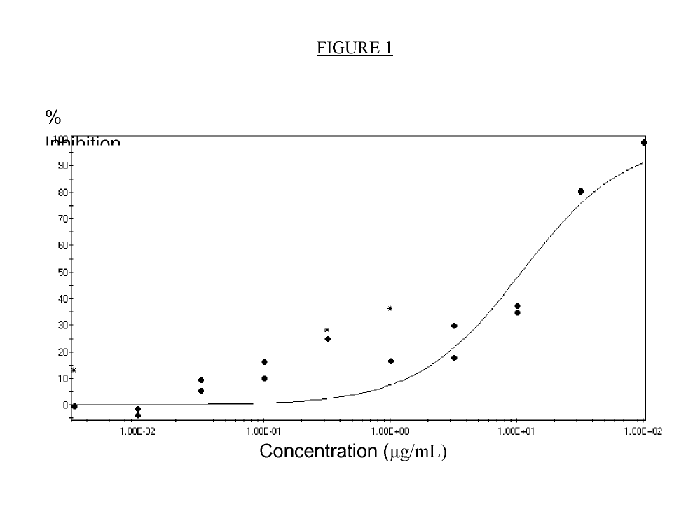

Serotonin uptake via the transporter was inhibited by the Nigella sativa extract I (from Example 1) in a dose dependent manner. The measured IC₅₀ values for inhibition of serotonin uptake by Nigella sativa extract is 14.14±4.5 μg/mL and is shown in FIG. 1. The data show that an lipophilic Nigella sativa seed extract may have mood lifting effects.

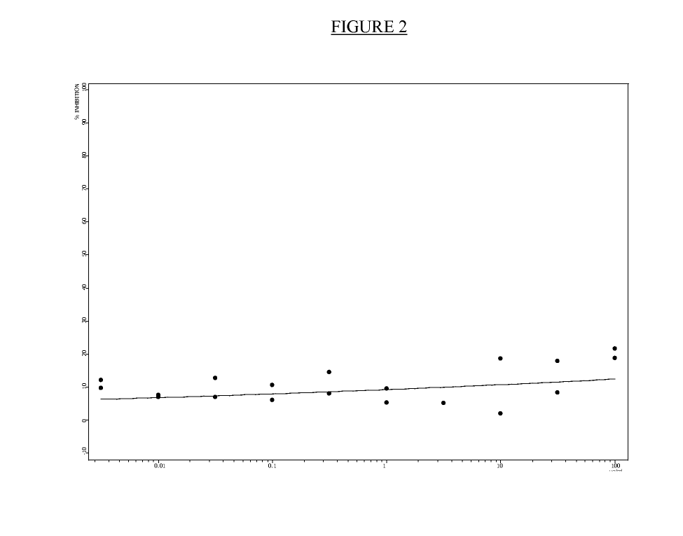

In contrast, a cold pressed oil of the seeds of black cumin (Abstswinder Schwarzkümmelöl, Germany) containing mostly polyunsaturated fatty acids and 0.3% (w/w) thymoquinone did not have this effect, yielding an IC50 of 129.4 μg/mL. (See FIG. 2)

### Example 3

### Preparation of a Soft Gelatin Capsule

A soft gelatin capsule may be prepared comprising the following ingredients:

| | Amount per |
|---|---|
| Ingredient | Capsule |
| Nigella sativa | 150 mg |
| extract | |
| Lecithin | 50 mg |
| Soy bean oil | 250 mg |

Two capsules per day for 3 months may be administered to a human adult for the aleviation of mild chronic low mood. To alleviate the winter blues, the capsules should be taken starting from autumn when the days get shorter (i.e. mid-September) for at least 6 months.

### Example 4

### Preparation of a Soft Gelatin Capsule

A soft gelatin capsule may be prepared comprising the following ingredients:

| | Amount per |
|---|---|
| Ingredient | Capsule |
| Nigella sativa extract | 200 mg |
| Evening primrose oil | 300 mg |
| Vitamin B₆ | 100 mg |

One capsule per day, preferably during the second half of the menstrual cycle, may be taken for 14 days for the alleviation of premenstrual syndrome (PMS) or other symptoms e.g. increased tension and irritability associated with the menstrual cycle.

### Example 5

Preparation of an enriched tomato juice

| Ingredient | Amount [g] |
|---|---|
| Nigella sativa Extract | 0.12 |
| Tomato juice ad | 1000 |

The juice contains ca. 30 mg Nigella sativa extract per serving (250 ml). As a strengthener and for general mental well-being 2 servings per day (240 ml) is recommended.

### Example 6

### Dry Dog Food

Commercial basal diet for dogs (e.g. Mera Dog “Brocken”, MERA-Tiernahrung GmbH, Marienstraβe 80-84, D-47625 Kevelaer-Wetten, Germany) is sprayed with a solution of Nigella sativa extract in an amount sufficient to administer to a subject a daily dose of 50 mg per kg body weight, based on the weight of the Nigella sativa extract or its volatile components concentrate. The food composition is dried to contain dry matter of about 90% by weight. For an average dog of 10 kg body weight to consume approx. 200 g dry feed per day, the dog food contains approx. 2500 mg Nigella sativa extract or its volatile components/kg food. For heavier dogs, the feed mix is prepared accordingly.

### Example 7

### Wet Cat Food

Commercial basal diet for cats (e.g. Happy Cat “Adult”, Tierfeinnahrung, Südliche Hauptstraβe 38, D-86517 Wehringen, Germany) is mixed with a solution of Nigella sativa extract or its volatile components in an amount sufficient to administer to a subject a daily dose of 100 mg per kg body weight, based on the weight of the dried Nigella sativa extract or its volatile components concentrate. For an average cat of 5 kg of body weight to consume approx. 400 g of wet food, the cat food contains 1250 mg/kg Nigella sativa extract. The food composition is dried to contain dry matter of about 90% by weight.

### Example 8

### Dog Treats Containing Nigella sativa Extract

Commercial dog treats (e.g. Mera Dog “Biscuit” for dogs as supplied by Mera Tiernahrung GmbH, Marienstrasse 80-84, 47625 Kevelaer-Wetten, Germany) are sprayed with a solution of Nigella sativa extract or its volatile components in an amount sufficient to administer to the treats 5-50 mg per g treats, based on the weight of the dried Nigella sativa extract or its volatile components concentrate. The food composition is dried to contain dry matter of about 90% by weight.

### Example 9

### Cat Treats Containing Nigella sativa Extract

Commercial cat treats (e.g. Whiskas Dentabits for cats as supplied by Whiskas, Masterfoods GmbH, Eitzer Str. 215, 27283 Verden/Aller, Germany) are sprayed with a solution of Nigella sativa extract or its volatile components in an amount sufficient to administer to the treats 5-50 mg per g treats, based on the weight of the dried Nigella sativa extract or its volatile components concentrate. The food composition is dried to contain dry matter of about 90% by weight.

### Example 10

Preparation of a broccoli soup

| Ingredient | Amount | |
|---|---|---|
| Broccoli | 300 | g |
| Onion powder | 14 | g |
| Garlic powder | 5 | g |
| Vegetable stock | 850 | g |
| Potato flakes | 50 | g |
| Powdered almonds | 20 | g |
| Salt | 10 | g |
| Citric acid | 0.5 | g |
| Nigella sativa extract | 0.5 | g |
| In total | 1250 | g |

Preparation:

The vegetable is cooked for 15-20 min, mixed and purreed with the vegetable stock. Then, the other ingredients are added and heated again, before the soup is filled into tins.

1 serving (250 g) contains 100 mg of Nigella sativa Extract.

### Example 11

Preparation of a Cracker with Pepper flavor

| | Ingredients | Quantity [g] |
|---|---|---|
| 1.0 | Dough | |
| 1.1 | Wheat flour type 550 | 540.0 |
| 1.2 | Skim milk powder | 30.0 |
| 1.3 | Icing sugar | 13.0 |
| 1.4 | Whey powder sweet | 12.0 |
| 1.5 | Yeast powder autolysed | 10.0 |
| 1.6 | Salt | 4.6 |
| 1.7 | Backing powder | 4.0 |
| 1.8 | Nigella sativa Extract | 0.4 |
| 1.9 | Malt Powder | 7.2 |
| 1.10 | Mixed Spices * | 10.0 |
| 2.0 | Water (RT) | 100.0 |
| 3.0 | Fat/Flavor Mixture | |
| 3.1 | Vegetable Fat (biscofin) | 94.0 |
| 3.2 | Flavor -PAPRIKA- 530214 E | 1.5 |
| | Guivaudan) | |
| 4.0 | Syrup Solution | |
| 4.1 | Water | 56.8 |
| 4.2 | Glucose Syrup DE 38 | 14.4 |
| 4.3 | Lactic Acid | 1.6 |
| 5.0 | Ammonium carbonate solution | |
| 5.1 | Water | 88.5 |
| 5.2 | Ammonium carbonate | 5.4 |
| 6.0 | Dusting Powder | |
| 6.1 | Wheat flour Type 550 | 82.0 |
| 6.2 | Salt fine ground | 0.6 |
| 6.3 | Vegetable fat | 17.4 |
| | Total (dough) | 1093.4 |

Mixed Spices

| * | Ingredients | Quantity [g] |
|---|---|---|
| 8.0 | Spice Mixture | |
| 8.1 | Onion powder | 2.50 |
| 8.2 | Garlic powder | 3.10 |
| 8.3 | White pepper | 0.775 |
| 8.4 | Oregano | 0.625 |
| 8.5 | Parsley | 0.625 |
| 8.6 | Nutmeg | 2.375 |
| | Total | 10.00 |

### Preparation of Dry Mixture and Solutions

Sieve all dry ingredients and add 1.1-1.10 into Kenwood type mixer
Mix the ingredients from position 3.0
Heat the water (4.0) and dissolve the glucose syrup, add lactic acid
Dissolve ammonium carbonate in water (5.1-5.2)

### Preparation of the Dough

Start the mixer before adding the water (2.0)
Add the other ingredients (3.0, 4.0, 5.0) bit by bit according to the ingredient list
Knead the dough for at least 5 min

### Form the Dough

Roll the dough in several layers (about 3) and sprinkle with dusting powder (6.0) between the layers
Roll to a final thickness of 1.5 mm
Prick holes into the dough and cut into pieces (ca. 2.5 cm×2.5 cm) of one serving size

### Baking and Drying

Bake the crackers at 220° C. for 5-7 min
Dry the crackers subsequent for 1,5 hours at 80°; cool at RT

100 g of the ready-to-eat crackers contains 47 mg Nigella sativa extract.

1. An oral dietary or nutraceutical composition comprising a lipophilic Nigella sp. seed extract in a effective amount for the treatment of symptoms connected to impaired or reduced neurotransmission in an animal.

2. The composition of claim 1 wherein the Nigella sp. seed extract is obtained by an extraction method selected from the group consisting of: liquid carbon dioxide under supercritical conditions, steam distillation/hydrodistillation yielding essential oils, ethanol extraction, ethyl acetate extraction, methyl-tert.-butylether, methanol, propane, butane, acetone and nitro oxide.

3. A dietary composition comprising a composition according to claim 1, selected from the group consisting of: fortified food, spicy cereal bars, bakery items, cookies, dietary supplements, non-alcoholic drinks, soft drinks, sport drinks, vegetable juices, teas, liquid food, and soups.

4. A composition according to claim 1 which is from Nigella sativa.

5. Use of a lipophilic Nigella sp. seed extract according to claim 1 for the manufacture of a composition for the treatment of a disorder connected to impaired neurotransmission in healthy people or animals.

6. Use according to claim 5, wherein the composition is a mood/vitality improver, a stress reliever, a condition improver, a reducer of anxiety, a reducer of tension, a reducer of unhappiness/discontentedness, a reducer of irritability, a reducer of dysphoria, a reducer of obsessive-compulsive behaviour, a relaxant, a sleep improver and/or an insomnia alleviator.

7. A use according to claim 5 wherein the composition prevents or normalizes circadian rhythm disruptions.

8. A use according to claim 5 which is a veternary use.

9. A use according to claim 5 wherein the Nigella sp. is Nigella sativa.

10. A method of treating a condition resulting from impaired neurotransmission comprising administering to an animal, including a healthy human, an effective amount of a lipophilic Nigella sativa seed extract or of at least one of its volatile components, and observing the reduction of imparied neurotransmission.

11. The method of claim 10 wherein the animal treated is selected from the group consisting of: farm animals, companion animals, aquaculture animals and animals used in the fur industry.

12. A method according to claim 10 wherein the condition is selected from the group consisting of: mood/vitality imbalance, a stress, tension, discontentedness, irritability, dysphoria, dysthymia, obsessive-compulsive behaviour, and insomnia.
